(19) United States
(12) Patent Application Publication
(10) Pub. No.: US 2025/0383273 A1
(43) Pub. Date: Dec. 18, 2025

(54) DEVICES FOR TESTING VOLUME DEFORMATIONS OF MASS CONCRETE

(71) Applicants: TIANJIN PORT ENGINEERING INSTITUTE CO., LTD. OF CCCC FIRST HARBOR ENGINEERING CO., LTD., Tianjin (CN); CCCC FIRST HARBOR ENGINEERING CO., LTD., Tianjin (CN); TIANJIN PORT ENGINEERING QUALITY TESTING CENTER CO., LTD., Tianjin (CN)

(72) Inventors: Xingang WANG, Tianjin (CN); Wei SI, Tianjin (CN); Zhijun CHEN, Tianjin (CN); Shiguang FAN, Tianjin (CN); Zhongchun SU, Tianjin (CN); Xin LIU, Tianjin (CN)

(73) Assignees: TIANJIN PORT ENGINEERING INSTITUTE CO., LTD. OF CCCC FIRST HARBOR ENGINEERING CO., LTD., Tianjin (CN); CCCC FIRST HARBOR ENGINEERING CO., LTD., Tianjin (CN); TIANJIN PORT ENGINEERING QUALITY TESTING CENTER CO., LTD., Tianjin (CN)

(21) Appl. No.: 19/315,440

(22) Filed: Aug. 29, 2025

Related U.S. Application Data
(63) Continuation of application No. PCT/CN2024/104952, filed on Jul. 11, 2024.

(30) Foreign Application Priority Data
Feb. 6, 2024 (CN) 202410168268.X

(51) Int. Cl. G01N 3/06; G01N 3/08; G01N 33/38

(52) U.S. Cl. CPC G01N 3/06; G01N 3/08; G01N 33/383

(57) ABSTRACT

Provided is a volumetric deformation testing device, which relates to the field of mass concrete testing. The volumetric deformation testing device includes a protective cylinder, a connecting block is provided in an inner cavity of the protective cylinder, a positioning rod is provided at an upper end of the connecting block, and a detecting assembly is provided at an upper end of the positioning rod. The detecting assembly is configured to monitor the volumetric deformation of the mass concrete, thereby facilitating the subsequent judgment of the shrinkage or expansion conditions of the mass concrete under different temperature conditions.

### CROSS-REFERENCE TO RELATED APPLICATIONS

This application is a Continuation of International Patent Application No. PCT/CN2024/104952, filed on Jul. 11, 2024, which claims priority to Chinese Patent Application No. 202410168268.X, filed on Feb. 6, 2024, the entire contents of each of which are incorporated herein by reference.

### TECHNICAL FIELD

The present disclosure relates to the field of mass concrete testing technology, and in particular, to a device for testing a volumetric deformation of mass concrete.

### BACKGROUND

Cement concrete is currently the most widely used material in civil engineering. Mechanical strength and volumetric stability are the most critical performance indicators affecting its engineering application. As is well known, a high strength of concrete is attributed to a hydration reaction between cement particles and water. The hydration reaction of cement requires the presence of liquid water and a relatively long curing period to achieve a design strength of the concrete. However, even in the presence of liquid water, a hydration rate of cement is significantly reduced under a low-temperature condition, resulting in a slow increase in the strength of the concrete. Therefore, when freshly mixed concrete is exposed to a low-temperature environment under conditions such as lack of pre-curing, insufficient pre-curing time, or sudden temperature drops, a cementitious structure has not yet fully formed, leading to the presence of a large amount of freezable water in the concrete. The cementitious structure at this stage lacks sufficient strength to resist an expansive force generated by the freezing of free water, which causes the formation and propagation of microcracks in the concrete, thereby adversely affecting the strength and long-term performance development of the concrete. To prevent early-age frost damage of concrete, numerous studies and practical applications have adopted preventive measures, such as incorporating an antifreeze agent to lower the freezing point of water, adding an early strength agent to accelerate cement hydration and strength development, applying external thermal insulation for curing, or providing an additional heat source to avoid or delay the freezing of the free water in the concrete.

Existing devices for testing volumetric deformations of concrete suffer from low accuracy, poor reliability, high testing costs, overly simplistic structure, and low automation. Moreover, such devices are incapable of accurately monitoring the actual volumetric deformation of concrete, making it difficult to precisely record the shrinkage or expansion behavior of the concrete. Therefore, it is desirable to provide a device for testing a volumetric deformation of mass concrete.

### SUMMARY

The purpose of the present disclosure is to provide a device for testing a volumetric deformation of mass concrete to solve the above-mentioned deficiencies in the existing technologies.

To realize the above purpose, the present disclosure provides the following technical solution:

A device for testing a volumetric deformation of mass concrete is provided. The device for testing a volumetric deformation of mass concrete includes a protective cylinder, a connecting block is provided in an inner cavity of the protective cylinder, a positioning rod is provided at an upper end of the connecting block, and a detecting assembly is provided at an upper end of the positioning rod, the detecting assembly is configured to monitor the volumetric deformation of the mass concrete; the detecting assembly includes a gear, a positioning block is provided at a central position of a lower end of the gear, a support plate is fixedly mounted at a lower end of the positioning block, a longitudinal cross-section of the support plate is in a pentagonal shape, and an adjusting member is slidably mounted on an inner wall of each end portion of the support plate, an upright rod is provided at a lower end of each of the adjusting members, and a flexible plate is sleeved over outer surfaces of the upright rods arranged at the lower ends of adjacent adjusting members; an end of the gear is uniformly provided with a plurality of arc-shaped grooves, and a sliding block is slidably mounted on an inner wall of each of the plurality of arc-shaped grooves; the adjusting member includes a movable plate, and an upper end of the movable plate is fixedly connected to a lower end of the sliding block; a snap-fit block is provided in an inner cavity of one end of the movable plate, and a detecting block is rotatably mounted at an end of the snap-fit block; an adjusting plate is slidably mounted on an outer surface of the detecting block, and an arc-shaped plate is fixedly mounted at an end of the adjusting plate away from the detecting block; and a transmission block is provided at an upper end of the support plate and located on a side of the gear, and an outer surface of the transmission block meshes with an outer surface of the gear.

In some embodiments, a recess is provided on a side of the detecting block, an inner wall of the recess is provided with a detecting member, an end of the detecting member away from the detecting block is connected with the adjusting plate, and an elastic member is sleeved over an outer surface of the detecting member.

Compared with the existing technologies, the device for testing the volumetric deformation of mass concrete provided in the present disclosure causes the sliding block fixedly mounted on the upper end of the movable plate to move when the adjusting member is actuated. Since the sliding block is slidably connected to the arc-shaped groove, the movement of the sliding block can synchronously drive the rotation of the gear about the positioning block. In addition, the gear is engaged with the transmission block such that, during the rotation of the gear, the transmission block is driven to rotate. By obtaining the rotation direction and rotation angle of the transmission block, the device is capable of recording the shrinkage or expansion behavior of the concrete.

By providing the detecting member, a pressure applied to the arc-shaped plate can be directly sensed, and a sliding distance of the adjusting plate relative to the detecting block can be determined based on the pressure applied to the arc-shaped plate, and then the volumetric deformation of the mass concrete can be determined based on the sliding distance of the adjusting plate, thereby avoiding errors introduced by a mechanical transmission chain of the gear and improving monitoring accuracy. Movement data of the adjusting plate may be transmitted to a control device in real time, and the control device may be configured to record a time point at which the mass concrete shrinks or expands. In addition, a temperature detector (e.g., a temperature sensor) may be provided on a side of the control device to record the temperature in the current environment, so that after the detecting member records the data, current environmental conditions are recorded simultaneously, thereby facilitating subsequent evaluation of the shrinkage or expansion behavior of the concrete under different temperature conditions.

By providing the elastic member, the adjusting plate can be automatically reset after an external force applied to the adjusting plate is removed, without the need for a manual adjustment, thereby improving the intelligence level of the device for testing the volumetric deformation of mass concrete, and avoiding mechanical jamming. In addition, a preloading force provided by the elastic member can prevent the detecting member from being displaced due to vibration or impact, making the device for testing the volumetric deformation of mass concrete suitable for harsh construction environments.

### BRIEF DESCRIPTION OF THE DRAWINGS

In order to more clearly illustrate the technical solutions in the embodiments of the present disclosure, the accompanying drawings that need to be used in the embodiments will be briefly introduced in the following. It is obvious that the following description of the accompanying drawings is only some of the embodiments recorded in the present disclosure, and other accompanying drawings can be obtained from these drawings by a person of ordinary skill in the art.

FIG. 1 is a schematic diagram illustrating an exemplary structure of a detecting assembly according to some embodiments of the present disclosure;

FIG. 2 is a longitudinal sectional view of an internal structure of a detecting assembly according to some embodiments of the present disclosure;

FIG. 3 is a transverse sectional view of an internal structure of a detecting assembly according to some embodiments of the present disclosure;

FIG. 4 is a schematic diagram illustrating an exemplary structure of an adjusting member according to some embodiments of the present disclosure;

FIG. 5 is a longitudinal sectional view of an internal structure of an adjusting member according to some embodiments of the present disclosure; and

FIG. 6 is a flowchart illustrating an exemplary process for testing a volumetric deformation of mass concrete according to some embodiments of the present disclosure.

### NUMERALS IN THE DRAWINGS

100, device for testing a volumetric deformation of mass concrete; 1, protective cylinder; 2, detecting assembly; 11, connecting block; 12, positioning rod; 13, cylinder side wall; 14, cylinder bottom wall; 21, gear; 211, transmission block; 22, arc-shaped groove; 23, sliding block; 24, positioning block; 25, support plate; 251, support base plate; 252, support protruding arm; 26, adjusting member; 27, arc-shaped plate; 28, upright rod; 29, flexible plate; 261, adjusting plate; 262, movable plate; 263, snap-fit block; 264, detecting block; 265, recess; 266, detecting member; 267, elastic member.

### DETAILED DESCRIPTION

To more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

In the description of the present disclosure, it should be noted that the terms “center,” “upper,” “lower,” “left,” “right,” “vertical,” “horizontal,” “inner,” “outer,” or the like indicate positional or orientation relationships based on the accompanying drawings. These terms are used merely for the purpose of facilitating the description and simplifying the explanation, and are not intended to indicate or imply that the referenced devices or components must have a particular orientation or be constructed and operated in a particular orientation. Therefore, such terms should not be construed as limiting the present disclosure. The terms “first,” “second,” “third,” and the like are used for descriptive purposes only and should not be interpreted as indicating or implying relative importance. Furthermore, unless otherwise explicitly stated or limited, the terms “mounted,” “connected,” and “coupled” are to be interpreted broadly. For example, the connection may be a fixed connection, a detachable connection, or an integral connection; it may be a mechanical connection or an electrical connection; it may be a direct connection or an indirect connection through an intermediate medium; it may also refer to communication between the internal portions of two components. Those skilled in the art may understand the specific meanings of the above terms in the context of the present disclosure based on the specific implementation.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

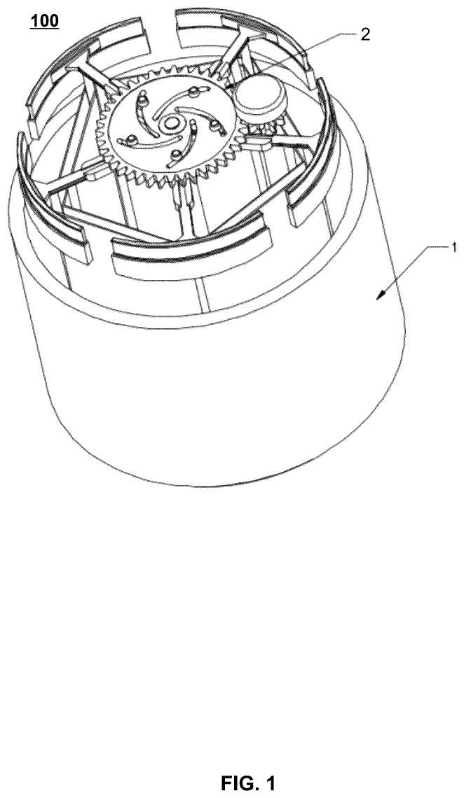

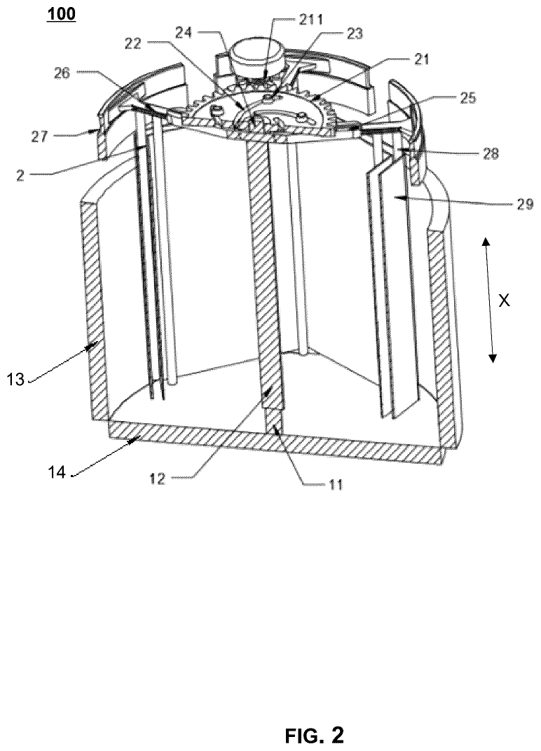

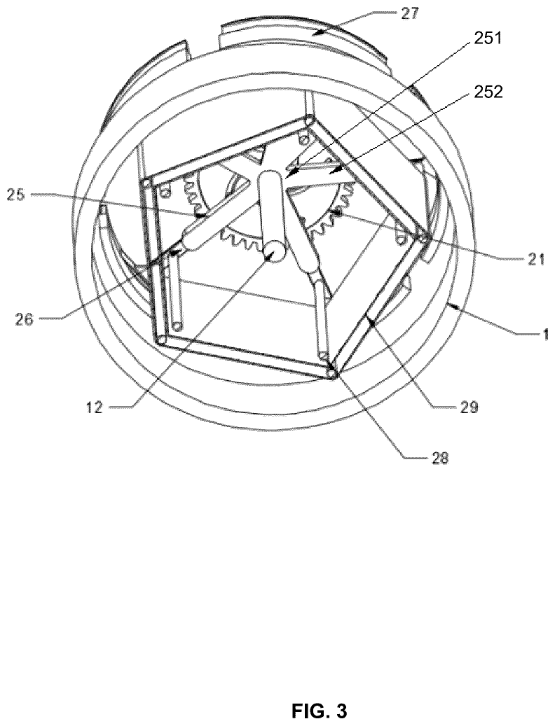

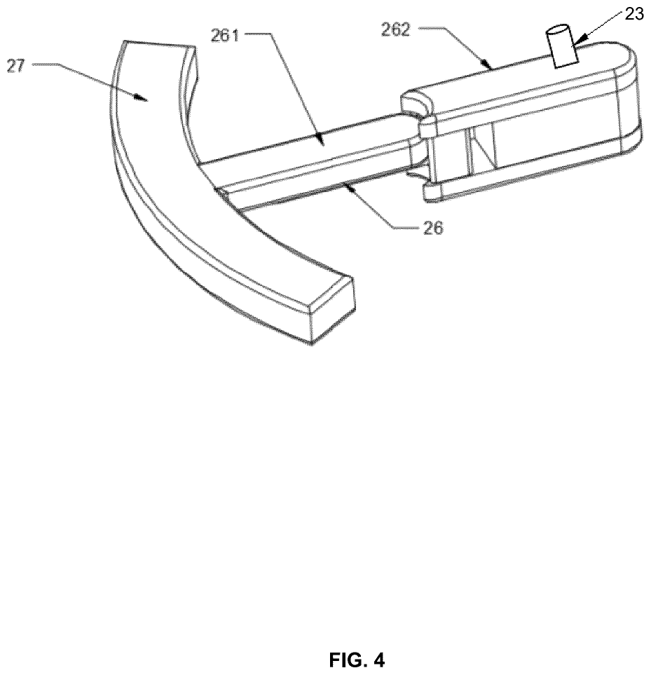

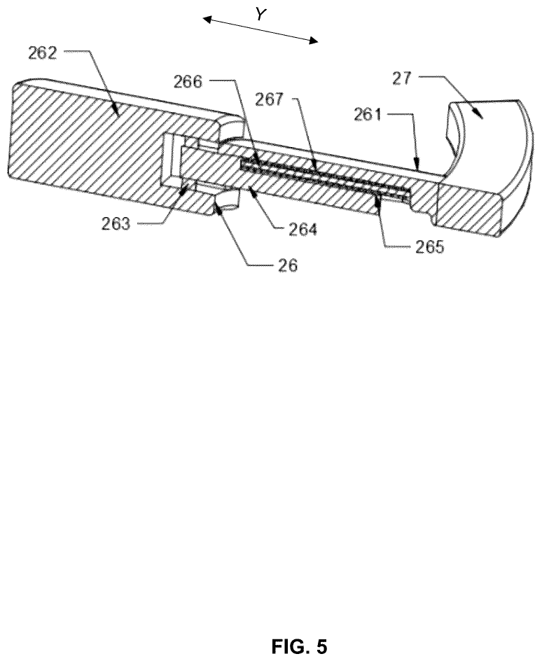

FIG. 1 is a schematic diagram illustrating an exemplary structure of a detecting assembly according to some embodiments of the present disclosure. FIG. 2 is a longitudinal sectional view of an internal structure of a detecting assembly according to some embodiments of the present disclosure. FIG. 3 is a transverse sectional view of an internal structure of a detecting assembly according to some embodiments of the present disclosure. FIG. 4 is a schematic diagram illustrating an exemplary structure of an adjusting member according to some embodiments of the present disclosure. FIG. 5 is a longitudinal sectional view of an internal structure of an adjusting member according to some embodiments of the present disclosure.

In some embodiments, as shown in FIG. 1 to FIG. 5, the device for testing the volumetric deformation of mass concrete 100 includes a protective cylinder 1. A connecting block 11 is provided in an inner cavity of the protective cylinder 1, a positioning rod 12 is provided at an upper end of the connecting block 11, and a detecting assembly 2 is provided at an upper end of the positioning rod 12. The detecting assembly 2 is configured to monitor the volumetric deformation of the mass concrete. The detecting assembly 2 includes a gear 21, and a positioning block 24 is provided at a central position of a lower end of the gear 21. A support plate 25 is fixedly mounted at a lower end of the positioning block 24. A longitudinal cross-section of the support plate 25 is in a pentagonal shape, and an adjusting member 26 is slidably mounted on an inner wall of each end portion of the support plate 25. An upright rod 28 is provided at a lower end of each of the adjusting members 26, and a flexible plate 29 is sleeved over outer surfaces of the upright rods 28 arranged at the lower ends of adjacent adjusting members 26. An end of the gear 21 is uniformly provided with a plurality of arc-shaped grooves 22, and a sliding block 23 is slidably mounted on an inner wall of each of the plurality of arc-shaped grooves 22. Each of the adjusting members 26 includes a movable plate 262, and an upper end of the movable plate 262 is fixedly connected to a lower end of one of the sliding blocks 23. A snap-fit block 263 is provided in an inner cavity at an end of each of the movable plates 262, and a detecting block 264 is rotatably mounted at an end of the snap-fit block 263. An adjusting plate 261 is slidably mounted on an outer surface of the detecting block 264, and an arc-shaped plate 27 is fixedly mounted at an end of the adjusting plate 261 away from the detecting block 264. A transmission block 211 is provided at an upper end of the support plate 25 and located on a side of the gear 21, and an outer surface of the transmission block 211 meshes with an outer surface of the gear 21.

The device for testing a volumetric deformation of mass concrete 100 (also referred to as the volumetric deformation testing device 100) refers to a device that is configured to detect the volumetric deformation of the mass concrete when the concrete shrinks or expands of the concrete. In some embodiments, the volumetric deformation testing device 100 applicable for detecting the volumetric deformation of mass concrete with a volume greater than 1 m³.

The protective cylinder 1 refers to a cylinder structure that accommodates the mass concrete to be detected. For example, the protective cylinder 1 includes a cylinder side wall 13 and a cylinder bottom wall 14. The cylinder bottom wall 14 is arranged at an end of the cylinder side wall 13 in a sealed manner to accommodate the mass concrete to be detected.

In some embodiments, as shown in FIG. 1 to FIG. 4, the connecting block 11 is provided in the inner cavity of the protective cylinder 1, the positioning rod 12 is provided at the upper end of the connecting block 11, and the detecting assembly 2 is provided at the upper end of the positioning rod 12. The detecting assembly 2 may be configured to monitor the volumetric deformation of the mass concrete accommodated in the protective cylinder 1.

The inner cavity of the protective cylinder 1 refers to a cavity formed by an inner wall of the protective cylinder 1, i.e., a cavity that accommodates the mass concrete. For example, the inner cavity of the protective cylinder 1 is a cavity enclosed by an inner surface of the cylinder side wall 13 and an inner surface of the cylinder bottom wall 14. The inner surface of the cylinder side wall 13/the cylinder bottom wall 14 refers a surface of the cylinder side wall 13/the cylinder bottom wall 14 that faces an interior of the protective cylinder 1. It should be noted that the connecting block 11 may be connected to an inner side wall (i.e., the inner surface of the cylinder side wall 13) of the protective cylinder 1, or the connecting block 11 may be connected to an inner bottom wall (i.e., the inner surface of the cylinder bottom wall 14) of the protective cylinder 1. In the embodiment illustrated in FIG. 2, the connecting block 11 is connected to the inner surface of the cylinder bottom wall 14 of the protective cylinder 1.

The connecting block 11 refers to a connecting member for fixedly connecting other components to the inner cavity of the protective cylinder 1.

In some embodiments, the connecting block 11 may be mounted in the inner cavity of the protective cylinder 1 and connected to other components in a fixed connection manner or a detachable connection manner. The fixed connection manner includes welding, riveting, one-piece molding, or the like. The detachable connection manner includes a magnetic connection, a threaded connection, or the like.

The upper end of the connecting block 11 refers to an end of the connecting block 11 away from the inner bottom wall of the protective cylinder 1 (i.e., the inner surface of the cylinder bottom wall 14) in a height direction of the protective cylinder 1. The height direction of the protective cylinder 1 may be indicated by the arrow X in FIG. 2. By way of example, in the embodiment illustrated in FIG. 2, the connecting block 11 is connected to the cylinder bottom wall 14 of the protective cylinder 1, and an end of the connecting block 11 where the connecting block 11 is connected to the cylinder bottom wall 14 may be referred to as a lower end of the connecting block 11. An end of the connecting block 11 away from the cylinder bottom wall 14 may be referred to as the upper end of the connecting block 11.

The positioning rod 12 is connected to the connecting block 11 in the same or similar manner as the connecting block 11 is connected to the protective cylinder 1. More descriptions regarding how the positioning rod 12 is connected to the connecting block 11 may be found elsewhere in the present disclosure (e.g., the connection manner between the connecting block 11 and the protective cylinder 1 and related descriptions thereof).

The positioning rod 12 refers to a structure for fixing a relative position between the detecting assembly 2 and the protective cylinder 1. For example, the positioning rod 12 may be a positioning cylinder, a positioning prism, or the like.

The detecting assembly 2 refers to a component that detects the volumetric deformation of the mass concrete when the concrete shrinks or expands of the mass concrete. More descriptions regarding the detection of the volumetric deformation of the mass concrete by the detecting assembly 2 may be found in the related descriptions below.

The upper end of the positioning rod 12 refers to an end of the positioning rod 12 away from the connecting block 11 in the height direction of the protective cylinder 1. Conversely, an end of the positioning rod 12 connected to the connecting block 11 may be referred to as a lower end of the positioning rod 12. More descriptions regarding the connection between the positioning rod 12 and the detecting assembly 2 may be found in the related descriptions below.

In some embodiments, the positioning rod 12 is provided with a sensor configured to detect an internal gas distribution within the mass concrete. For example, the positioning rod 12 may be a hollow cylinder, and a sensor (e.g., a fiber-optic gas sensor, etc.) for detecting the internal gas distribution of the mass concrete is pre-embedded in the hollow cylinder to achieve the function of detecting the internal gas distribution within the mass concrete.

In the embodiments of the present disclosure, by installing a device (e.g., the sensor) with a detecting function in the inner cavity of the positioning rod 12, the internal gas distribution within the mass concrete can be detected, thereby improving the monitoring accuracy and monitoring effect of the volumetric deformation testing device 100. In some embodiments, the sensor that is provided in the positioning rod 12 may be communicatively connected to a control device and transmits acquired sensing data to the control device in real time, thereby improving an automation degree of the volumetric deformation testing device 100.

More descriptions regarding the control device may be found in the related descriptions below.

In some embodiments, the detecting assembly 2 includes the gear 21, the positioning block 24 is provided at the central position of the lower end of the gear 21, and the support plate 25 is fixedly mounted at the lower end of the positioning block 24. The longitudinal cross-section of the support plate 25 is in the pentagonal shape, and an adjusting member 26 is slidably mounted on the inner wall of each end portion of the support plate 25. One or more upright rods 28 are provided at the lower end of each adjusting member 26, and a flexible plate 29 is sleeved over the outer surfaces of the upright rods 28 arranged at the lower ends of two adjacent adjusting members 26.

In some embodiments, the detecting assembly 2 is configured such that when the mass concrete shrinks or expands, the gear 21 rotates, and the detecting assembly 2 determines the volumetric deformation of the mass concrete based on a rotation direction and a rotation angle of the gear 21. More descriptions regarding determining the volumetric deformation of the mass concrete based on the rotation direction and the rotation angle of the gear 21 may be found in the related descriptions below.

In some embodiments, the gear 21 may include a cylindrical gear, a bevel gear, or the like.

The lower end of the gear 21 refers to an end of the gear 21 facing the inner bottom wall (i.e., the inner surface of the cylinder bottom wall 14) of the protective cylinder 1 when a rotation axis of the gear 21 is parallel to the height direction of the protective cylinder 1. In contrast, when the rotation axis of the gear 21 is parallel to the height direction of the protective cylinder 1, an end of the gear 21 that is away from the inner bottom wall of the protective cylinder 1 is an upper end of the gear 21. The central position refers to a rotation center of the gear 21. It may be understood that the rotation center of the gear 21 is positioned on the rotation axis of the gear 21.

The positioning block 24 refers to a central shaft component that provides a rotational fulcrum for the gear 21. For example, the positioning block 24 may be a positioning cylinder.

In some embodiments, the positioning block 24 may be provided at the central position of the lower end of the gear 21 to support the gear 21 and provide the rotational fulcrum for the gear 21. For example, a matching groove is provided at the rotation center of the lower end of the gear 21, and the positioning block 24 may be matched with the matching groove, so that the gear 21 may rotate around the positioning block 24. As another example, a through-hole is provided at the rotation center of the gear 21, and the positioning block 24 passes through the through-hole from the lower end of the gear 21. The positioning block 24 is in clearance fit with the through-hole, so that the gear 21 may rotate around the positioning block 24.

In some embodiments, a through-hole is provided at the rotation center of the gear 21, the positioning block 24 passes through the through-hole from the lower end of the gear 21. A spacer plate is provided at an upper end of the positioning block 24, and the spacer plate is located at the upper end of the gear 21. The isolation plate is used to confine the positioning block 24, thereby preventing the positioning block 24 from disengaging from the gear 21.

More descriptions regarding the Isolation plate may be found in the related descriptions below.

The lower end of the positioning block 24 refers to an end of the positioning block 24 facing the inner bottom wall of the protective cylinder 1 (i.e., the inner surface of the cylinder bottom wall 14) in the height direction of the protective cylinder 1. In contrast, the upper end of the positioning block 24 refers to an end of the positioning block 24 away from the inner bottom wall of the protective cylinder 1 in the height direction of the protective cylinder 1.

The support plate 25 refers to a plate-shaped structure for supporting the detecting assembly 2.

In some embodiments, the support plate 25 is configured to support the detecting assembly 2 on the positioning rod 12. For example, the upper end of the positioning rod 12 and the lower end of the positioning block 24 are respectively connected to two ends of the support plate 25 to support the detecting assembly 2 on the positioning rod 12. As another example, a positioning through-hole is provided at a center of the support plate 25. The lower end of the positioning block 24 passes through the positioning through-hole and is directly connected to the upper end of the positioning rod 12. A cross-sectional dimension of the positioning rod 12 is larger than an aperture of the positioning through-hole. The cross-sectional dimension of the positioning rod 12 refers to the maximum dimension of a cross section that is perpendicular to the height direction of the positioning rod 12. For example, when the cross section is a circle, the cross-sectional dimension is the diameter of the circle. As another example, when the cross-section is in a square, the cross-sectional dimension is the diagonal length of the square. In this embodiment, since the cross-sectional dimension of the positioning rod 12 is larger than the aperture of the positioning through-hole, the upper end of the positioning rod 12 may abut against the support plate 25, so that the support plate 25 can support the detecting assembly 2 on the positioning rod 12.

The longitudinal cross-section of the support plate 25 refers to a cross section of the support plate 25 that is perpendicular to a thickness direction of the support plate 25. In the embodiment illustrated in FIG. 1 to FIG. 3, the thickness direction of the support plate 25 is parallel to the height direction of the protective cylinder 1 (as shown by the arrow X in FIG. 2).

The longitudinal cross-section of the support plate 25 is in a pentagonal shape refers to that the longitudinal cross-section of the support plate 25 has a shape similar to a five-pointed star. For ease of understanding, as illustrated by FIG. 3, the support plate 25 includes a support base plate 251 and five support protruding arms 252 arranged around the support base plate 251. The support base plate 251 is fixedly mounted to the positioning block 24. The five support protruding arms 252 are connected to the support base plate 251 and extend radially outward from the support base plate 251. An end of each of the five support protruding arms 252 away from the support base plate 251 is referred to as an end portion of the support plate 25. It may be understood that in the longitudinal cross-section (i.e., the cross-section of the support plate 25 that is perpendicular to the thickness direction of the support plate 25) of the support plate 25, the ends of the five support protruding arms 252 away from the support base plate 251 may be approximated regarded as five vertices, and the shape of the entire longitudinal cross-section may be approximately regarded as a five-pointed star.

In some embodiments, a plurality of support protruding arms 252 are evenly spaced along a peripheral side of the support base plate 251.

It should be noted that a count of the support protruding arms 252 is not limited to five as shown in FIG. 1 to FIG. 3. The count of the support protruding arms 252 may be adjusted according to actual requirements, for example, three, four, six, or more. Correspondingly, the shape of the longitudinal cross-section of the support plate 25 varies based on the count of the support protruding arms 252. For example, if the count of the support protruding arms 252 is four, the shape of the longitudinal cross-section of the support plate 25 may be approximated regarded as a four-pointed star.

In some embodiments, the sliding mounting between the adjusting member 26 and the support plate 25 may be realized in a plurality of ways. For example, a sliding groove is provided at each end portion of the support plate 25, and the adjusting member 26 may be placed directly in the sliding groove and is able to move relative to the sliding groove. As another example, the sliding groove is provided at each end portion of the support plate 25, a slide rail is provided in the sliding groove, and the adjusting member 26 is provided with a pulley adapted to the slide rail, to realize the sliding mounting of the adjusting member 26.

The adjusting member 26 refers to a structure that drives the gear 21 to rotate when the mass concrete shrinks or expands. More descriptions regarding the adjusting member 26 driving the gear 21 to rotate may be found in the related descriptions below.

The lower end of the adjusting member 26 refers to an end of the adjusting member 26 near the inner bottom wall of the protective cylinder 1 (i.e., the inner surface of the cylinder bottom wall 14) after the adjusting member 26 is mounted on the support plate 25.

The adjacent adjusting members 26 refer to two adjusting members 26 slidably mounted on two adjacent end portions of the support plate 25.

More descriptions regarding the adjusting members 26 may be found elsewhere in the present disclosure (e.g., FIG. 5 and related descriptions thereof).

The upright rod 28 refers to a rod-shaped structure provided along the height direction of the protective cylinder 1. For example, the upright rod 28 may be a cylinder or a prism.

The outer surface of the upright rod 28 refers to an outer surface of a sidewall of the upright rod 28. For example, if the upright rod 28 is a solid rod, the outer surface of the upright rod 28 is the surface of the sidewall of the upright rod 28. As another example, if the upright rod 28 is a hollow rod (e.g., a sensor is provided inside the upright rod), the outer surface of the upright rod 28 is the outermost surface of the sidewall of the upright rod 28.

The flexible plate 29 refers to a plate-shaped structure made of a flexible material. The flexible material may include a composite material, synthetic rubber, or the like.

In some embodiments, the flexible plate 29 is configured to wrap the mass concrete so as to prevent leakage before the concrete solidifies. A plurality of flexible plates 29 are constraint by all of the upright rods 28 provided at the lower ends of the adjusting members 26, such that after the flexible plate 29 wraps the concrete, the flexible plate 29 remains stable without shaking. In this embodiment, a flexible plate 29 is sleeved over the outer surfaces of the upright rods 28 arranged at the lower ends of every two adjacent adjusting members 26, so that a plurality of flexible plates 29 cooperates with the cylinder bottom wall 14 of the protective cylinder 1 to form a sealed cavity. The sealed cavity wraps the mass concrete, so that the mass concrete does not leak and shake before it solidifies.

In some embodiments, the shrinkage or expansion of the mass concrete may be recorded through the upright rod 28. For example, a device (e.g., a sensor) with a detection function is provided inside the upright rod 28, and the device with the detection function may be configured to detect the internal gas distribution within the mass concrete.

The term “sleeved” refers to the flexible plate 29 covering the outer surfaces of two upright rods 28 respectively, the two upright rods 28 being arranged at the lower ends of two adjacent adjustment members 26, so as to achieve fixation of the flexible plate 29.

In some embodiments, as shown in FIG. 3, a flexible plate 29 is sleeved over the outer surfaces of two upright rods 28 arranged at the lower ends of every two adjacent adjusting members 26 to form a double-layer plate structure. A layer of the double-layer plate structure is located on a side of the two upright rods 28 away from the support plate 25. Another layer of the double-layer plate structure is located on a side of the two upright rods 28 near the support plate 25. By designing the double-layer plate structure, the wrapping effect on the mass concrete is improved.

In some embodiments, at least one upright rod 28 is provided at the lower end of each of the adjusting members 26, and a flexible plate 29 is sleeved over the outer surfaces of two upright rods 28 arranged at the lower ends of every two adjacent adjusting members 26. In other embodiments, at least two upright rods 28 are provided at the lower end of each adjusting member 26, and a flexible plate 29 is sleeved over the outer surfaces of two upright rods 28 arranged at the lower ends of two adjacent adjusting members 26. For example, in the embodiment illustrated in FIG. 3, a first upright rod and a second upright rod are provided at the lower end of each adjusting member 26, and the first upright rod and the second upright rod are arranged along a length direction of the each adjusting member 26. The second upright rod is located on a side of the first upright rod away from the support plate 25, and the flexible plate 29 is sleeved over the outer surfaces of the two second upright rods 28 arranged at the lower ends of adjacent adjusting members 26. It may be understood that, in addition to sleeving the flexible plate 29 over the outer surfaces of the two second upright rods arranged at the lower ends of adjacent adjusting members 26, the flexible plate 29 may also be provided at the outer surfaces of the two first upright rods arranged at the lower ends of the adjacent adjusting members 26, to form a multi-layer plate structure, thereby further improving the wrapping effect on the mass concrete.

In some embodiments, an end of the gear 21 is uniformly provided with a plurality of arc-shaped grooves 22, and a sliding block 23 is slidably mounted on the inner wall of each of the plurality of arc-shaped grooves 22.

An arc-shaped groove 22 refers to a groove extending along an arc. For example, each of the plurality of arc-shaped grooves 22 may be a groove that extends along a circular-arc segment. In some embodiments, an end of each of the plurality of arc-shaped grooves 21 points to the rotation center of the gear 21, and another end of each of the plurality of arc-shaped grooves 21 is located away from the rotation center of the gear 21.

The end of the gear 21 refers to the upper end of the gear 21 or the lower end of the gear 21. The end of the gear 21 being provided with the plurality of arc-shaped grooves 22 refers to that the plurality of arc-shaped grooves 22 are formed from the upper end of the gear 21 and extend through to the lower end of the gear 21, or the plurality of arc-shaped grooves 22 are formed from the lower end of the gear 21 and extend through to the upper end of the gear 21.

The end of the gear being uniformly provided with the plurality of arc-shaped grooves 22 refers to that the plurality of arc-shaped grooves 22 are arranged at equal intervals around the rotation center of the gear 21. For example, included angles between lines connecting proximal ends of adjacent arc-shaped grooves 22 to the rotation center of the gear 21 are the same. The proximal end of an arc-shaped groove 22 refers to an end of the arc-shaped groove 22 that is near the rotation center of the gear 21. In contrast, a distal end of an arc-shaped groove 22 refers to an end of the arc-shaped groove 22 that is away from the rotation center of the gear 21. It should be understood that, in the embodiment shown in FIG. 1, the gear 21 is uniformly provided with five arc-shaped grooves 22, and the included angle between the two lines respectively connecting the proximal ends of the adjacent arc-shaped grooves 22 to the rotation center of the gear 21 is 72°.

It should be understood that to convert the movement of the adjusting members 26 into the rotation of the gear 21, a count and a distribution of the plurality of arc-shaped grooves 22, a shape of the support plate 25 (e.g., a count and distribution of the support protruding arms 252), and a count and a distribution of the adjusting members 265 have a corresponding relationship. For example, the count of the arc-shaped grooves 22, the count of the support protruding arms 252, and the count of the adjusting members 26 are the same, the support protruding arms 252 are evenly spaced along the peripheral side of the support base plate 251, and the arc-shaped grooves 22 are uniformly distributed. Each adjusting member 26 corresponds to one support protruding arm 252 and one arc-shaped groove 22. When an adjusting member 26 moves relative to the corresponding support protruding arm 252, the gear 21 is driven (by the cooperation between the arc-shaped groove 22 and the sliding block 23 described below) to rotate by the arc-shaped groove 22.

Based on the above principle, the count of the arc-shaped grooves 22 is not limited to the five shown in FIG. 1, but corresponds to the shape of the support plate 25 (e.g., the count of the support protruding arms 252). For example, if the count of the support protruding arms 252 is four and the shape of the longitudinal cross section of the support plate 25 is approximated as a four-pointed star, the count of arc-shaped grooves 22 is four.

The sliding block 23 refers to a sliding member provided in the arc-shaped groove 22 and capable of moving along the arc-shaped groove 22. In some embodiments, the sliding block 23 is configured to be connected to the adjusting member 26, and when the mass concrete shrinks or expands, the adjusting member 26 slides relative to the support plate 25 under the action of the mass concrete, thereby driving the sliding block 23 to move relative to the support plate 25. The sliding block 23 may drive the gear 21 to rotate when the sliding block 23 moves, thereby detecting the volumetric deformation of the mass concrete.

In some embodiments, the adjusting member 26 includes a movable plate 262, and an upper end of the movable plate 262 is fixedly connected to a lower end of the sliding block 23.

The movable plate 262 refers to a structure that drives the sliding block 23 to move relative to the support plate 25 under the action of the mass concrete when the concrete shrinks or expands.

In some embodiments, the movable plate 262 is configured to be slidably mounted to the end portion of the support plate 25, so that the adjusting member 26 may slide relative to the support plate 25. More descriptions regarding the sliding mounting may be found in the related descriptions above.

The upper end of the movable plate 262 refers to an end of the movable plate 262 away from the inner bottom wall of the protective cylinder 1 (i.e., the inner surface of the cylinder bottom wall 14) in the height direction of the protective cylinder 1. In contrast, the lower end of the movable plate 262 refers to an end of the movable plate 262 close to the inner bottom wall of the protective cylinder 1 in the height direction of the protective cylinder 1. The lower end of the sliding block 23 refers to an end of the sliding block 23 near the inner bottom wall of the protective cylinder 1 in the height direction of the protective cylinder 1. In contrast, an upper end of the sliding block 23 refers to an end of the sliding block 23 away from the inner bottom wall of the protective cylinder 1 in the height direction of the protective cylinder 1.

In the embodiment of the present disclosure, since the sliding block 23 is connected to the movable plate 262, when the movable plate 262 slides relative to the support plate 25, the sliding block 23 is driven to move, and then the sliding block 23 drives the gear 21 to rotate around the positioning block 24.

It may be understood that when the adjusting member 26 moves in a direction away from the support plate 25, the adjusting member 26 drives the sliding block 23 to slide from the proximal end of the arc-shaped groove 22 to the distal end of the arc-shaped groove 22, and drives the gear 21 to rotate along a first direction. The greater the distance the adjusting member 26 moves, the greater the rotation angle of the gear 21 is. When the adjusting member 26 moves in a direction toward the support plate 25, the adjusting member 26 drives the sliding block 23 to slide from the distal end of the arc-shaped groove 22 to the proximal end of the arc-shaped groove 22, and the gear 21 is driven to rotate in a direction opposite to the first direction. The greater the distance the adjusting member 26 moves, the greater the rotation angle of the gear 21 is. Therefore, when the mass concrete shrinks or expands, the adjusting member 26 moves under the action of the mass concrete, and the volumetric deformation of the mass concrete can be monitored based on the rotation direction and rotation angle of the gear 21.

In some embodiments, the snap-fit block 263 is provided in an inner cavity at an end of the movable plate 262, and the detecting block 263 is rotatably mounted at an end of the snap-fit block 264.

The inner cavity at the end of the movable plate 262 refers to a cavity formed at an end of the movable plate 262 away from the positioning block 24. For example, the inner cavity at the end of the movable plate 262 may be a cavity formed by a snap-fit groove provided on an end surface of the end portion of the movable plate 262 away from the positioning block 24.

The snap-fit block 263 refers to a snap-fit member configured to secure the detecting block 264 in the inner cavity at the end of the movable plate 262. For example, the snap-fit block 263 may be a snap-fit protrusion formed in the inner cavity at the end of the movable plate 262, which mates with a snap-fit slot provided on the detection block 264 to retain the detecting block 264 within the inner cavity at the end of the movable plate 262.

The detecting block 264 refers to a structure configured to detect the volumetric deformation of the mass concrete when the concrete shrinks or expands. More descriptions regarding the detecting block 264 may be found elsewhere in the present disclosure (e.g., FIG. 5 and related descriptions thereof).

In some embodiments, the detecting block 264 is rotatably connected to the snap-fit block 263, so that the detecting block 264 may rotate around the snap-fit block 263 relative to the movable plate 262, i.e., the snap-fit block 263 serves as a rotation axis of the detecting block 264. For example, in the embodiment illustrated in FIG. 5, the snap-fit block 263 may be a snap-fit cylinder extending from a lower end of the inner cavity at the end of the movable plate 262 to an upper end of the inner cavity at the end of the movable plate 262. A snap-fit hole adapted to the snap-fit block 263 is formed at an end of the detecting block 264. The detecting block 264 is snap-fitted in the inner cavity at the end the movable plate 262 via a snap-fit cooperation between the snap-fit hole and the snap-fit cylinder, and the detecting block 264 is capable of rotating around the snap-fit block 263, thereby rotating relative to the movable plate 262.

In some embodiments, the adjusting plate 261 is slidably mounted on the outer surface of the detecting block 264, and the arc-shaped plate 27 is fixedly mounted at the end of the adjusting plate 261 away from the detecting block 264.

The outer surface of the detecting block 264 refers to a surface of the detecting block 264 that is outside the inner cavity at the end of the movable plate 262.

The sliding mounting manner between the adjusting plate 261 and the detecting block 264 is the same as or similar to the sliding mounting manner between the adjusting member 26 and the support plate 25. More descriptions regarding the sliding mounting manner of the adjusting plate 261 and the detecting block 264 may be found elsewhere in the present disclosure (e.g., the sliding mounting manner of the adjusting member 26 and the support plate 25 and related descriptions thereof).

The end of the adjusting plate 261 away from the detecting block 264 refers to an end of the adjusting plate 261 that is farthest from the detecting block 264 in a sliding direction of the adjusting plate 261. For example, in the embodiment illustrated in FIG. 5, the sliding direction of the adjusting plate 261 may be indicated by the arrow Y. The end of the adjusting plate 261 away from the detecting block 264 refers to an end of the adjusting plate 261 that is farthest from the detecting block 264 in the direction of the arrow Y.

The arc-shaped plate 27 refers to a plate-shaped structure having a curvature. For example, the arc-shaped plate 27 may be a wave-shaped plate or a circular arc plate.

In some embodiments, the arc-shaped plate 27 is configured to drive the movable plate 262 to slide under the action of the mass concrete, thereby driving the gear 21 to rotate through the sliding block 23 to monitor the volumetric deformation of the mass concrete when the concrete shrinks or expands. For example, the arc-shaped plate 27 extends into the interior of the mass concrete, and the arc-shaped plate 27 drives the adjusting plate 261 to slide toward the movable plate 262 under the action of the mass concrete when the concrete shrinks or expands. When the adjusting plate 261 slides to a first limiting position, the adjusting plate 261 drives the detecting block 264 and the movable plate 262 to move toward the support plate 25, thereby driving the sliding block 23 to move, and finally driving the gear 21 to rotate along the first direction via the sliding block 23. When the mass concrete expands, the arc-shaped plate 27 drives the adjusting plate 261 to slide away from the movable plate 262 under the action of the mass concrete. When the adjusting plate 261 slides to a second limiting position, the adjusting plate 261 drives the detecting block 264 and the movable plate 262 to move away from the support plate 25, thereby driving the sliding block 23 to move, and finally driving the gear 21 to rotate in a direction opposite to the first direction via the sliding block 23. The first limiting position and the second limiting position refer to two end points of a sliding range of the adjusting plate 261. The first limiting position may be an end point of the sliding range of the adjusting plate 261 near the movable plate 262, and the second limiting position may be an end point of the sliding range of the adjusting plate 261 away from the movable plate 262.

In some embodiments, the rotation of the gear 21 is achieved through contact the mass concrete and the arc-shaped plate 27. The curved surface design of the arc-shaped plate 27 not only adapts to the shape of the protective cylinder 1 but also ensures that the arc-shaped plate 27 is in uniform contact with the mass concrete when the mass concrete shrinks or expands, thereby reducing point-contact stress concentration and preventing localized damage to the arc-shaped plate 27. In addition, the curved surface design enables the arc-shaped plate 27 to conform to the complex surface of the mass concrete and adapt to non-planar deformation of the mass concrete, thereby ensuring high-precision, high-applicability, and long-lifespan monitoring.

In some embodiments, the transmission block 211 is provided at the upper end of the support plate 25 and located on the side of the gear 21, and the outer surface of the transmission block 211 meshes with the outer surface of the gear 21.

The transmission block 211 being provided at the upper end of the support plate 25 and located on the side of the gear 21 means that the transmission block 211 is provided on a side of the upper end of the support plate 25 near the gear 21. The outer surface of the gear 21 refers to a surface on which gear teeth are provided. The outer surface of the transmission block 211 refers to a surface of the transmission block 211 that is capable of meshing with the gear teeth.

In the embodiment of the present disclosure, when the adjusting member 26 moves, the adjusting member 26 drives the sliding block 23 fixedly mounted on the upper end of the movable plate 262 to move. Since the sliding block 23 is slidably mounted on the arc-shaped groove 22, when the sliding block 23 moves, the movement of the sliding block 23 synchronously drives the gear 21 to rotate around the positioning block 24. At the same time, the outer surface of the gear 21 meshes with the transmission block 211, so that when the gear 21 rotates, the gear 21 drives the transmission block 211 to rotate. The shrinkage or expansion conditions of the concrete is recorded based on a rotation direction and a rotation angle of the transmission block 211.

In some embodiments, the upper end of the transmission block 211 is provided with a sensor (e.g., an angle sensor) for detecting the rotation direction and the rotation angle of the transmission block 211. In some embodiments, the sensor provided on the transmission block 211 is connected to an external control device, and the sensor may transmit the rotation angle of the transmission block 211 to the external control device.

The volumetric deformation testing device 100 provided in the embodiments of the present disclosure is provided with the sliding block 23, the arc-shaped groove 22, the adjusting member 26, and the arc-shaped plate 27, so that when the arc-shaped plate 27 drives the adjusting member 26 to move, the adjusting member 26 may drive the sliding block 23 fixedly mounted on the upper end of the adjusting member 26 to move. Since the outer surface of the sliding block 23 is slidably mounted on the inner wall of the arc-shaped groove 22, when the sliding block 23 moves, the sliding block 23 can synchronously drive the gear 21 to rotate around the positioning block 24, and the volumetric deformation of the mass concrete is monitored by recording the rotation direction and the rotation angle of the gear 21. In addition, the outer surface of the gear 21 meshes with the transmission block 211, so that when the gear 21 rotates, the gear 21 drives the transmission block 211 to rotate, and the shrinkage or expansion conditions of the concrete can be recorded by recording the rotation direction and the rotation angle of the transmission block 211.

In some embodiments, as shown in FIG. 5, a recess 265 is provided on a side of the detecting block 264, and an inner wall of the recess 265 is provided with a detecting member 266. An end of the detecting member 266 away from the detecting block 264 is connected to the adjusting plate 261, and an elastic member 267 is sleeved over an outer surface of the detecting member 266.

The side of the detecting block 264 refers to a portion of the outer surface of the detecting block 264. For example, as shown in FIG. 5, the side of the detecting block 264 is an upper side of the outer surface of the detecting block 264 (i.e., a side of the outer surface of the detecting block 264 that is away from the inner bottom wall of the protective cylinder 1).

The detecting member 266 refers to a device configured to detect a pressure value applied by the mass concrete to the arc-shaped plate 27.

In some embodiments, the detecting member 266 is configured to detect a sliding distance of the adjusting plate 261 relative to the detecting block 264 when the mass concrete shrinks or expands. For example, the detecting member 266 is a device with a telescopic detection function, such as a pressure sensor. It should be understood that the detecting member 266 is provided in the recess 265, and the end of the detecting member 266 away from the detecting block 264 is connected to the adjusting plate 261, so that when the mass concrete shrinks or expands, the arc-shaped plate 27 can drive the adjusting plate 261 to compress or stretch the detecting member 266. The detecting member 266 generates a corresponding detection value (e.g., a pressure value of the pressure sensor) when the adjusting plate 261 is compressed or stretched. A telescoping stroke of the detecting member 266 may be determined based on the detection value of the detecting member 266 (e.g., the pressure value of the pressure sensor), and the telescoping stroke of the detecting member 266 may be regarded as the sliding distance of the adjusting plate 261.

The clastic member 267 refers to an elastic component (e.g., a spring) configured to provide a restoring force for returning the adjusting plate 261 to an initial position after movement. The initial position refers to a position of the adjusting plate 261 when the arc-shaped plate 27 is not subjected to a force exerted by the mass concrete (e.g., when the mass concrete is not accommodated in the protective cylinder 1, or when the mass concrete has not shrunk or expanded). In some embodiments, the clastic member 267 is sleeved over the outer surface of the detecting member 266, an end of the clastic member 267 is connected to the adjusting plate 261, and another end of the elastic member 267 is connected to the detecting block 264.

In this embodiment, when the mass concrete shrinks, the adjusting member 261 compresses the detecting member 266 and the clastic member 267. After an external force acting on the adjusting member 261 disappears (e.g., when the mass concrete is removed from the protective cylinder 1), the elastic member 267 may push the adjusting member 261 to the initial position. When the mass concrete expands, the adjusting member 261 stretches the detecting member 266 and the clastic member 267. When the external force acting on the adjusting member 261 disappears, the clastic member 267 may pull the adjusting member 261 o the initial position.

In some embodiments, the detecting member 266 may be connected to the external control device and transmit movement data of the adjusting plate 261 to the control device in real time. The control device may be configured to record a time point when the mass concrete shrinks or expand. In some embodiments, the control device may be provided with a temperature detector (e.g., a temperature sensor) for recording a temperature in a current environment, so that when the detecting member 266 records data, conditions of the current environment is recorded simultaneously, thereby facilitating subsequent analysis of the shrinkage or expansion conditions of the mass concrete under different temperature conditions. More descriptions regarding the detecting member 266 being connected to the external control device to achieve real-time data transmission may be found in the related descriptions below

In some embodiments of the present disclosure, by providing the detecting member 266, the pressure exerted on the arc-shaped plate 27 when the concrete shrinks or expands can be directly sensed. Based on the pressure exerted on the arc-shaped plate 27, the sliding distance of the adjusting plate 261 relative to the detecting block 264 can be determined. Then the volumetric deformation of the mass concrete is determined based on the sliding distance of the adjusting plate 261, thereby avoiding errors introduced by the mechanical transmission chain of the gear 21 and improving monitoring accuracy. By providing the elastic member 267, the adjusting plate 261 can automatically return to the initial position after the external force applied to the adjusting plate 261 disappears, which eliminates the need for a manual adjustment, thereby improving the intelligence degree of the volumetric deformation testing device 100, and avoiding mechanical jamming. In addition, the preloading force provided by the elastic member 267 can prevent the detecting member 266 from being displaced due to vibration or impact, making the volumetric deformation testing device 100 suitable for harsh construction environments.

In some embodiments, a control device may be provided and configured to work in conjunction with electrical components in the present disclosure. The control device may be any type of controller connected to the electrical components in the present disclosure, and configured to control operational states (e.g., switching on and off) of the electrical components. By way of example, a single-chip microcomputer (SCM) may serve as the control device. The SCM is a typical embedded microcontroller unit (MCU), comprising an arithmetic unit, a controller, a storage device, and an input/output (I/O) interface-essentially functioning as a microcomputer. Compared with a general-purpose microprocessor used in a personal computer, the embedded MCU places more emphasis on self-supplying (no need for external hardware) and cost savings. Advantages of the embedded MCU include a compact size, an ability to be installed inside instruments, simple I/O interfaces, and low power consumption. More descriptions regarding the connection between the control device and the electrical components, and how the control device controls the operation of the electrical components may be found in the related descriptions above.

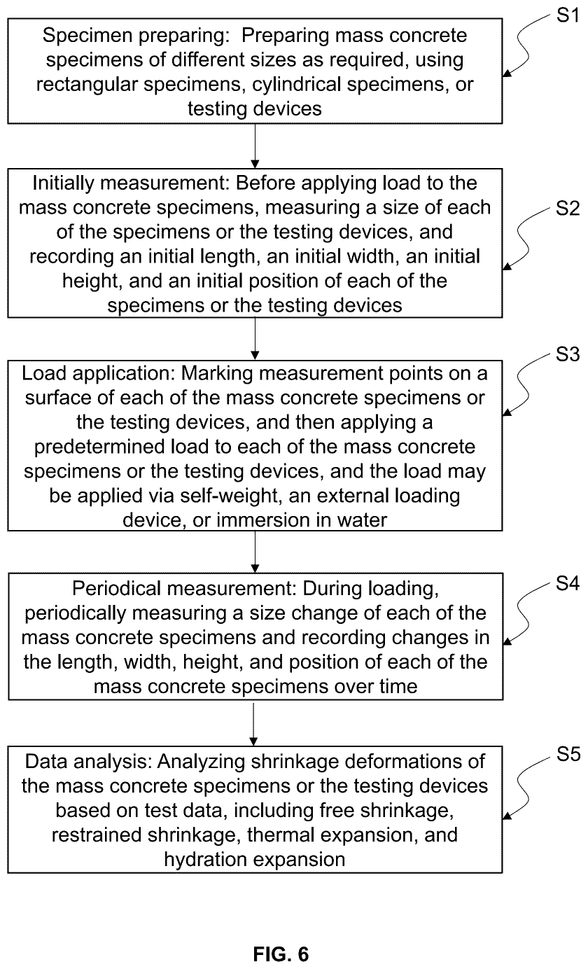

The present disclosure further provides a method for testing a volumetric deformation of mass concrete. FIG. 6 is a flowchart illustrating an exemplary process of a method for testing a volumetric deformation of mass concrete according to some embodiments of the present disclosure. In some embodiments, the method for testing the volumetric deformation of the mass concrete includes the following steps:

S1, specimen preparing. Preparing mass concrete specimens of different sizes as required, using rectangular specimens, cylindrical specimens, or testing devices.

S2, initial measurement. Before applying load to the mass concrete specimens, measuring a size of each of the specimens or the testing devices, and recording an initial length, an initial width, an initial height, and an initial position of each of the specimens or the testing devices.

S3, load application. Marking measurement points on a surface of each of the mass concrete specimens or the testing devices, and then applying a predetermined load to each of the mass concrete specimens or the testing devices, and the load may be applied via self-weight, an external loading device, or immersion in water.

S4, periodic measurement. During loading, periodically measuring a size change of each of the mass concrete specimens and recording changes in the length, width, height, and position of each of the mass concrete specimens over time.

S5, data analysis. Analyzing shrinkage deformations of the mass concrete specimens or the testing devices based on test data, including free shrinkage, restrained shrinkage, thermal expansion, and hydration expansion.

In summary, the present disclosure achieves at least the following technical effects:

First, the volumetric deformation testing device provided in the embodiments of the present disclosure is provided with the sliding block, the arc-shaped groove, the adjusting member, and the arc-shaped plate. When the arc-shaped plate drives the adjusting member to move, the adjusting member can drive the sliding block that is fixedly mounted on the upper end of the adjusting member. Since the outer surface of the sliding block is slidably mounted on the inner wall of the arc-shaped groove, when the sliding block moves, the sliding block can synchronously drive the gear to rotate around the positioning block. The volumetric deformation of the mass concrete is monitored by recording the rotation direction and the rotation angle of the gear. In addition, the outer surface of the gear meshes with the transmission block, so that when the gear rotates, the gear drives the transmission block to rotate, and the shrinkage or expansion conditions of the mass concrete is recorded by recording the rotation direction and the rotation angle of the transmission block.

Second, the volumetric deformation testing device provided in the embodiments of the present disclosure is provided with the detecting member that can directly sense the pressure exerted on the arc-shaped plate when the concrete shrinks or expands. Based on the pressure exerted on the arc-shaped plate, the sliding distance of the adjusting plate relative to the detecting block can be determined. Then the volumetric deformation of the mass concrete can be determined based on the sliding distance of the adjusting plate, thereby avoiding errors introduced by the mechanical transmission chain of the gear and improving monitoring accuracy.

Thirdly, the volumetric deformation testing device provided in the embodiments of the present disclosure is provided with the elastic member, which enable the adjusting plate to automatically return to the initial position after the external force applied to the adjusting plate disappears, without manual adjustment, thereby improving the intelligence degree of the volumetric deformation testing device and preventing mechanical jamming. In addition, the preloading force provided by the clastic member can prevent the detecting member from being displaced due to vibration or impact, making the volumetric deformation testing device suitable for harsh construction environments.

The basic concepts are described above. Obviously, for those skilled in the art, the above detailed disclosure is only an example, and does not constitute a limitation to the present disclosure. Although not expressly stated here, those skilled in the art may make various modifications, improvements, and corrections to the present disclosure. Such modifications, improvements and corrections are suggested in present disclosure, so such modifications, improvements, and corrections still belong to the spirit and scope of the exemplary embodiments of the present disclosure.

Meanwhile, the present disclosure uses specific words to describe the embodiments of the present disclosure. For example, “one embodiment,” “an embodiment,” and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This manner of disclosure does not, however, imply that the subject matters of the disclosure requires more features than are recited in the claims. Rather, claimed subject matters may lie in less than all features of a single foregoing disclosed embodiment.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present disclosure, or any of same that may have a limiting affect as to the broadest scope of the claims now or later associated with the present disclosure. By way of example, should there be any inconsistency or conflict between the description, definition, and/or the use of a term associated with any of the incorporated material and that associated with the present disclosure, the description, definition, and/or the use of the term in the present disclosure shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A device for testing a volumetric deformation of mass concrete, comprising a protective cylinder, wherein a connecting block is provided in an inner cavity of the protective cylinder, a positioning rod is provided at an upper end of the connecting block, and a detecting assembly is provided at an upper end of the positioning rod, the detecting assembly is configured to monitor the volumetric deformation of the mass concrete; wherein
the detecting assembly includes a gear, a positioning block is provided at a central position of a lower end of the gear, a support plate is fixedly mounted at a lower end of the positioning block, a longitudinal cross-section of the support plate is in a pentagonal shape, and an adjusting member is slidably mounted on an inner wall of each end portion of the support plate, one or more upright rods are provided at a lower end of each of the adjusting members, and a flexible plate is sleeved over outer surfaces of upright rods arranged at lower ends of every two adjacent adjusting members;
an end of the gear is uniformly provided with a plurality of arc-shaped grooves, and a sliding block is slidably mounted on an inner wall of each of the plurality of arc-shaped grooves;
each of the adjusting members includes a movable plate, and an upper end of the movable plate is fixedly connected to a lower end of one of the sliding blocks;
a snap-fit block is provided in an inner cavity at an end of the movable plate, and a detecting block is rotatably mounted at an end of the snap-fit block;
an adjusting plate is slidably mounted on an outer surface of the detecting block, and an arc-shaped plate is fixedly mounted at an end of the adjusting plate away from the detecting block; and
a transmission block is provided at an upper end of the support plate and located on a side of the gear, and an outer surface of the transmission block meshes with an outer surface of the gear.

2. The device of claim 1, wherein a recess is provided on a side of the detecting block, an inner wall of the recess is provided with a detecting member, an end of the detecting member away from the detecting block is connected to the adjusting plate, and an elastic member is sleeved over an outer surface of the detecting member.
